(19) United States
(12) Patent Application Publication
(10) Pub. No.: US 2015/0238376 A1
(43) Pub. Date: Aug. 27, 2015

(54) ADHESIVE BANDAGE DISPENSING DEVICE

(71) Applicant: Jody Seibold, Springfield, OH (US)

(72) Inventor: Jody Seibold, Springfield, OH (US)

(21) Appl. No.: 14/186,058

(22) Filed: Feb. 21, 2014

(51) Int. Cl. A61F 15/00

(52) U.S. Cl. CPC A61F 15/002

(57) ABSTRACT

An adhesive bandage dispensing device comprising a housing, where the housing includes an edging, a removable front face and a back face; a strip of adhesive bandages composed of a backing and multiple adhesive bandages; a first spool attached to the back face within the housing, where the strip of adhesive bandages wraps around the first spool; a first slot on the edging of the housing, where the first slot enables the strip of adhesive bandages to exit the housing; a second slot positioned to receives the backing after the adhesive bandage is removed; a second spool to recoil the backing after passing through the second slot; an actuator to initiate the strip out of the housing; and a motor coupled to the actuator and the second spool, and where the motor drives the second spool to advance the strip of adhesive bandages through the first slot.

### BACKGROUND OF THE INVENTION

1. Field of the Invention

The present invention relates to a dispenser for adhesive bandages that contains the bandages on a spool to dispense and discard of the backing in a single container.

2. Description of Related Art

Adhesive bandages are small dressings applied to non-serious wounds, cuts and scrapes. They are routinely used by medical personnel and childcare workers. In a hospital, very time blood is drawn, a shot is administered or a small wound is dressed a bandage is applied. Likewise, whenever a child falls and gets a scrape or cut an adhesive bandage is applied not only for the physical wound but also the emotional one. Nurses often find that repeated bandage application is time consuming and tedious, taking valuable time unwrapping the small packages when they'd prefer to be helping patients. Also, sterility may be difficult to maintain if multiple bandages have to be opened at once.

Therefore it would be beneficial in the art to provide a way to quickly unwrap and apply an adhesive bandage. It would also be desirable in the art to provide a device that maintains bandage sterility during application.

### SUMMARY OF THE INVENTION

In view of the foregoing disadvantages inherent in the prior art, the general purpose of the present invention is to provide an adhesive bandage dispenser, configured to include all of the advantages of the prior art, and to overcome the drawbacks inherent therein.

Accordingly, an object of the present invention is to provide a dispenser for adhesive bandages that automatically dispenses the bandage without the backing attached.

Another object of the present invention is to provide an adhesive bandage dispensing device that includes a motion activated sensor.

Yet another object of the present invention is to provide an adhesive bandage dispensing device that is battery powered for optimal portability.

To achieve the above objects, in an aspect of the present invention, an adhesive bandage dispensing device is described comprising a housing, where the housing includes an edging, a removable front face and a back face; a strip of adhesive bandages composed of a backing and multiple adhesive bandages; a first spool attached to the back face within the housing, where the strip of adhesive bandages wraps around the first spool; a first slot on the edging of the housing, where the first slot enables the strip of adhesive bandages to exit the housing; a second slot positioned to receives the backing after the adhesive bandage is removed; a second spool to recoil the backing after passing through the second slot; an actuator to initiate the strip out of the housing; and a motor coupled to the actuator and the second spool, and where the motor drives the second spool to advance the strip of adhesive bandages through the first slot.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1A depicts a strip of adhesive bandages in accordance with the present invention;

FIG. 1B depicts a perspective view of an adhesive bandage dispensing device in accordance with an exemplary embodiment of the present invention; and

FIG. 2 depicts a perspective view of an inner cavity of an adhesive bandage dispensing device in accordance with an exemplary embodiment of the present invention.

Like reference numerals refer to like parts throughout the description of several views of the drawings.

### DETAILED DESCRIPTION OF THE DRAWINGS

The present invention relates to a dispenser for adhesive bandages that contains the bandages on a spool to dispense and discard of the backing in a single container. The present invention provides an adhesive bandage dispensing device to assist users in removing adhesive bandages quickly while also maintaining sterility. The adhesive bandage dispensing device is an enclosed casing that incorporates two internal spools. A first spool that holds an elongated strip of adhesive bandages, and a second spool that holds the bandage backing The adhesive bandage dispensing device may include a small motor to drive the second spool in automatically dispensing the adhesive bandages. With the adhesive bandage dispensing device the user is provided the ability to unwrap the bandages quickly without compromising the bandage's sterility.

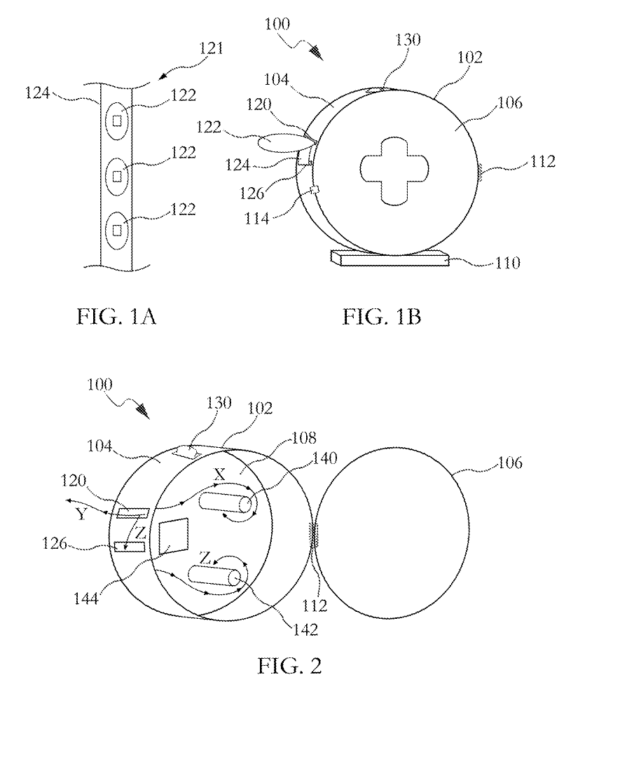

Turning now descriptively to the drawings, referring to FIG. 1, a perspective view of an adhesive bandage dispensing device 100 is shown in accordance with an exemplary embodiment of the present invention. The adhesive bandage dispensing device 100 is comprised of a housing 102 that may be shaped like a short cylinder, or like a hockey puck. Alternatively, the housing 102 may be cuboidal. The housing 102 includes an edging 104, a front face 106, and a back face, element 108 illustrated in FIG. 2. The front face 106 may be removable or it may open from the housing 102 on a hinge 112. A latch 114 may be positioned on a peripheral edge of the front face 106 to lift the front face 106 from the housing 102. The housing 102 may be made from aluminum or plastic to provide a lightweight, portable storage container.

The housing 102 may include a removable base 110. The base 110 provides a stable platform upon which the housing 102 rests. Alternately, the housing 102 may be mounted to a wall or cabinet to keep it out of the way, and away from contaminants. A portable housing 102 may be available to enable users, especially parents with small children, to carry the adhesive bandage dispensing device 100 in their purse, car or diaper bag.

Along the edging 104 may be a first slot 120 through which a strip of adhesive bandages 121 passes. The strip of adhesive bandages 121 is illustrated in FIG. 1(A), wherein the strip 121 includes a backing 124 and a plurality of adhesive bandages 122. The backing 124 may be made from paper or plastic, and is a continuous strip of material. The adhesive bandages 122 may be plastic, fabric, rubber or the like. The housing 102 and the strip of adhesive bandages 121 may accommodate adhesive bandages 122 of varying sized lengths and widths.

The strip of adhesive bandages 121 is sterile within the housing 102 to ensure that when dispensed the adhesive bandage 122 may be immediately applied to a cut or wound. After the strip 121 passes through the first slot 120, the adhesive bandage 122 is removed from the backing 124. The backing 124 then goes back into the housing 102 through a second slot 126. The adhesive bandage 122 lifts automatically from the backing 124 upon exiting the first slot 120.

Atop the housing 102 may be an actuator 130 to initiate dispensing the adhesive bandages 122. The actuator 130 may be positioned on the edging 104 or on the front face 106. The actuator 130 may be a button which is pressed when a new adhesive bandage 122 is desired. Additionally, the actuator 130 may include a sensor which automatically advances the strip of adhesive bandages 121 through the first slot when motion activated. For example, a user may wave their hand in front of the actuator 130 to dispense the adhesive bandage. This way a fresh, sterile bandage 122 is ready to apply with a simple hand wave by the user.

Referring now to FIG. 2, a perspective view of an inner cavity of the adhesive bandage dispensing device 100 is shown with the front face 106 lifted in accordance with an exemplary embodiment of the present invention. For clarity, the housing 102 is illustrated without the strip of adhesive bandages. Attached to the back face 108 of the housing 102 are a first spool 140 and a second spool 142. The housing 102 further includes a motor 144 which is the driving force to rotate the second spool 142, thereby dispensing the strip of adhesive bandages. The motor 144 is coupled to the actuator 130 and the second spool 142. The motor 144 may be electrically powered by plugging the housing 102 into a power outlet. Alternatively, the motor 144 may include batteries for portable use.

When the actuator 130 is activated it sends a signal to the motor 144 which then rotates the second spool 142. The second spool 142 is the driving force to dispense the strip of adhesive bandages from the first slot 120, and then recoil the backing around the second spool 142 after bandage removal. The strip of adhesive bandages is initially wrapped around the first spool 140. The strip follows arrow X around the first spool 140 and out of the first slot 120. The strip then splits, removing the adhesive bandage following arrow Y, and then rewrapping the backing by going back into the second slot 126 following arrow Z around the second spool 142. This way there is no trash to remove after dispensing the bandage and the strip is automatically driven with the backing as it is rewrapped around the motorized second spool 142. The motor 144 drives the second spool 142 to move a predetermined length of the strip through the first slot 120. The predetermined length coordinates with the length of the bandage being dispensed.

The adhesive bandage dispensing device 100 is an ideal product for hospitals, nursing homes, clinics, offices, daycares, and homes. The housing 102 automatically dispenses one adhesive bandage 122 at a time to provide a quickly available, unwrapped, sterile bandage as needed by the user. This may be especially useful for nursing staff that typically unwrap and apply numerous bandages a day. Also, parents and guardians will find the convenience of an automatic bandage dispenser without any trash especially useful when quickly trying to bandage a crying child.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. An adhesive bandage dispensing device comprising:
a. a housing, where the housing includes an edging, a removable front face and a back face;
b. a strip of adhesive bandages within the housing, where the strip of adhesive bandages includes a backing upon which a plurality of adhesive bandages are attached;
c. a first spool attached to the back face within the housing, where the strip of adhesive bandages wraps around the first spool;
d. a first slot on the edging of the housing, where the first slot enables the strip of adhesive bandages to exit the housing;
e. a second slot positioned below the first slot on the edging, where the second slot receives the backing after the adhesive bandage is removed;
f. a second spool attached to the back face within the housing, where the second spool recoils the backing after passing through the second slot;
g. an actuator fastened to the housing, where the actuator initiates the strip of adhesive bandages to exit the housing; and
h. a motor within the housing, where the motor is coupled to the actuator and the second spool, and where the motor drives the second spool to advance the strip of adhesive bandages through the first slot thereby recoiling the backing around the second spool.

2. The adhesive bandage dispensing device according to claim 1, where the front face is attached to the housing on a hinge, where the hinge enables the front face to open and close.

3. The adhesive bandage dispensing device according to claim 1, where the front face includes a latch to remove the front face from the housing.

4. The adhesive bandage dispensing device according to claim 1, where the actuator is a button, where when the button is pressed the strip of adhesive bandages dispenses the adhesive bandage.

5. The adhesive bandage dispensing device according to claim 1, where the actuator is a sensor, where the sensor is motion activated to dispense the adhesive bandage from the housing.

6. The adhesive bandage dispensing device according to claim 1, where the housing includes a base upon which to rest the housing.

7. The adhesive bandage dispensing device according to claim 1, where the housing is mounted to a structure.

8. The adhesive bandage dispensing device according to claim 1, where the motor is electrically powered.

9. The adhesive bandage dispensing device according to claim 1, where the motor is battery powered.

10. The adhesive bandage dispensing device according to claim 1, where the housing comprises a cylindrical shape.
